(19) United States
(12) Patent Application Publication
(10) Pub. No.: US 2019/0355472 A1
(43) Pub. Date: Nov. 21, 2019

(54) COMPUTER-IMPLEMENTED SYSTEM AND METHODS FOR PREDICTING THE HEALTH AND THERAPEUTIC BEHAVIOR OF INDIVIDUALS USING ARTIFICIAL INTELLIGENCE, SMART CONTRACTS AND BLOCKCHAIN

(71) Applicant: John D. Kutzko, Pagosa Springs, CO (US)

(72) Inventor: John D. Kutzko, Pagosa Springs, CO (US)

(21) Appl. No.: 16/415,597

(22) Filed: May 17, 2019

Related U.S. Application Data
(60) Provisional application No. 62/673,719, filed on May 18, 2018.

(51) Int. Cl. G16H 50/30; G16H 10/60

(52) U.S. Cl. CPC G16H 50/30; G16H 10/60

(57) ABSTRACT

A method of predicting the health and therapeutic behavior of patients may include: receiving a patient's healthcare data that includes an existing condition, a new condition, a limiting factor, and a compliance record for the existing condition; determining a therapeutic behavior pattern of patient using the compliance record for the existing condition; determining a successful therapy for the new condition based on the therapeutic behavior pattern; and calculating a cost quote for the successful therapy for a time period based on the limiting factor. A method of providing cost effective therapy for a patient may include: receiving healthcare data of a patient having a new condition; determining a successful therapy for the new condition; calculating a probability of disease progression for the new condition; determining a possible therapy for the new condition; calculating a cost quote for the possible therapy; and creating a smart contract for the possible therapy.

### CROSS REFERENCE TO RELATED APPLICATIONS

This application claims priority to and the benefit of the filing date of U.S. Provisional Application No. 62/673,719, filed on May 18, 2018, entitled “COMPUTER-IMPLEMENTED SYSTEM AND METHODS FOR PREDICTING THE HEALTH AND THERAPEUTIC BEHAVIOR OF INDIVIDUALS USING ARTIFICIAL INTELLIGENCE, SMART CONTRACTS AND BLOCKCHAIN”, which is hereby incorporated by reference in its entirety.

### FIELD OF THE INVENTION

This patent specification relates to the field of systems and methods for providing cost effective healthcare that is individually personalized.

### BACKGROUND

The health of individuals taking medications is impacted by a variety of factors. These factors may include the therapeutic behavior of the medications on the individual, synergistic or antagonistic behavior of other medications taken alongside an existing medication, co-morbidities, genetics, and lifestyle behaviors, such as smoking, weight changes, medication compliance, and mental health situations. Currently, there are no systems and methods which are able to use these factors to predict changes in the one or more conditions of an individual as well as to provide a prediction to achieve a healthier lifestyle through changes in lifestyle circumstances, changes in drug types, and information on interactions with other drugs that the individual is taking. Patients and healthcare providers, such as large employers, third party administrators, governmental agencies (DoD, Tricare, Va.) pharmacy benefit managers and insurance providers, would greatly benefit from systems and methods which can provide or predict the drug usage for an individual and which are also able to outline a drug usage and cost schedule for a period of time which will clearly define the healthcare needs of the individual.

Therefore, a need exists for novel computer-implemented systems and methods for providing cost effective healthcare that is individually personalized. A further need exists for novel computer-implemented systems and methods that are configured to use a plurality of patient specific factors to predict changes in the one or more conditions of an individual as well as to provide a prediction to achieve a healthier lifestyle through changes in lifestyle circumstances. There is also a need for novel computer-implemented systems and methods that are configured to provide or predict the drug usage for an individual and which are also able to outline a drug usage and cost schedule for a period of time which will clearly define the healthcare needs of the individual.

### BRIEF SUMMARY OF THE INVENTION

Computer-implemented system and methods for predicting the health and therapeutic behavior of individuals using artificial intelligence, smart contracts, and a blockchain database are provided. The system and methods disclosed herein may use a blockchain database of a blockchain network to enable a novel pharmacy benefits management healthcare model, preferably through creating Smart Healthcare contracts, to predict medication usage and spending of patients over time. In further embodiments, patients may benefit as tokenized/cryptocurrency is available for anonymized data collection and data maintenance which may eliminate copayments and coinsurances due from the patient. In still further embodiments, shared anonymized data of the system may be purchased by healthcare providers, such as pharma and health plan providers, to undergird the cryptocurrency coin/token value.

According to one embodiment consistent with the principles of the invention, a computer implemented method of predicting the health and therapeutic behavior of patients is provided. In some embodiments, the method may include the steps of: receiving healthcare data of a patient, via a client device, the healthcare data including an existing condition, a new condition, a limiting factor, and a compliance record for the existing condition; determining, via a computing device processor, a therapeutic behavior pattern of patient using the compliance record for the existing condition; determining, via the computing device processor, a successful therapy for the new condition based on the therapeutic behavior pattern; and calculating, via the computing device processor, a cost quote for the successful therapy for a time period based on the limiting factor.

According to another embodiment consistent with the principles of the invention, a computer implemented method of providing cost effective therapy for a patient is provided. In some embodiments, the method may include the steps of: receiving healthcare data of a patient, via a client device, the healthcare data including a new condition; determining, via a computing device processor, at least one successful therapy for the new condition; calculating, via the computing device processor, a probability of disease progression for the new condition; determining, via the computing device processor, at least one possible therapy for the new condition; calculating, via the computing device processor, a cost quote for at least one possible therapy; and creating, via the computing device processor, a smart contract for the at least one possible therapy.

It is an object of the present invention to provide a healthcare system that utilizes AWL, blockchain and cryptocurrency technologies.

It is an object of the present invention to provide a computer system that utilizes AWL, blockchain and cryptocurrency technologies that understands an individual healthcare needs and recommends alternative solutions.

It is an object of the present invention to provide a computer system that utilizes AWL, blockchain and cryptocurrency technologies that understands healthcare needs and recommends cost effective healthcare plans that can be used by the individual or healthcare professionals.

It is an object of the present invention to provide a computer system that utilizes AWL, blockchain and cryptocurrency technologies that takes data from individuals who are experiencing similar healthcare care needs and outcomes and creates healthcare strategies (drug, lifestyle, exercise) that may improve the healthcare of all or some of those target group individuals.

It is an object of the present invention to provide a computer system that utilizes AWL, blockchain and cryptocurrency technologies that takes data from individuals who are experiencing similar healthcare needs and predicts healthcare outcomes for that individual over a fixed time period—say 1 year.

It is an object of the present invention to provide a computer system that utilizes AWL, blockchain and cryptocurrency technologies that takes data from individuals who are experiencing similar healthcare needs and predicts healthcare outcomes for that individual over a fixed time period—say 1 year and works out the cost of fulfilling the healthcare needs of the individual over that period.

It is an object of the present invention to provide a computer system that utilizes AWL, blockchain and cryptocurrency technologies that takes data from individuals who are experiencing similar healthcare needs and predicts healthcare outcomes for that individual over a fixed time period—say 1 year and works out the cost of fulfilling the healthcare needs of the individual over that period and ensures that those costs do not increase over that period by helping the individual to follow the plan and learn to recommend solutions to improve their health.

It is an object of the present invention to provide a computer system that utilizes AWL, blockchain and cryptocurrency technologies that incentivize individuals to improve their health through rewarding them with set health goals that are linked to smart contracts.

It is an object of the present invention to provide a computer system that utilizes AWL, blockchain and cryptocurrency technologies that takes anonymized data from patients and identifies trends and adherence behaviours of drugs and services that can be used to improve pharmaceutical manufacturers understanding of the efficacy and adherence of their drug products.

It is an object of the present invention to provide a computer system that utilizes AWL, blockchain and cryptocurrency technologies that takes anonymized data from patients around the world and helps physicians, hospital and healthcare practitioners to identify better healthcare strategies for their patients.

### BRIEF DESCRIPTION OF THE DRAWINGS

Some embodiments of the present invention are illustrated as an example and are not limited by the figures of the accompanying drawings, in which like references may indicate similar elements and in which:

FIG. 1-FIG. 1 depicts an illustrative example of some of the components and computer implemented methods which may be found in a system for predicting the health and therapeutic behavior of individuals according to various embodiments described herein.

FIG. 2-FIG. 2 illustrates a block diagram showing an example of a server which may be used by the system as described in various embodiments herein.

FIG. 3-FIG. 3 shows a block diagram illustrating an example of a client device which may be used by the system as described in various embodiments herein.

FIG. 4A-FIG. 4A depicts a block diagram illustrating some applications of a system for predicting the health and therapeutic behavior of individuals which may function as software rules engines according to various embodiments described herein.

FIG. 4B-FIG. 4B shows a block diagram illustrating some components of a blockchain database according to various embodiments described herein

FIG. 5-FIG. 5 illustrates a block diagram of an example of a computer-implemented method of predicting the health and therapeutic behavior of patients according to various embodiments described herein.

FIG. 6-FIG. 6 shows a block diagram of an example of a computer-implemented method of providing cost effective therapy for a patient according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Although the terms “first”, “second”, etc. are used herein to describe various elements, these elements should not be limited by these terms. These terms are only used to distinguish one element from another element. For example, the first element may be designated as the second element, and the second element may be likewise designated as the first element without departing from the scope of the invention.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

Definitions

As used herein, the term “Patient” may refer to a person or animal under health care. The person or animal may be waiting for this care or may be receiving it or may have already received it. Examples of the definition of patient include; a person or animal who requires medical care; a person or animal receiving medical or dental care or treatment; a person or animal under a physician's or veterinarian's care for a particular disease or condition; a person or animal who is waiting for or undergoing medical treatment and care; and an individual or animal who is receiving needed professional services that are directed by a licensed practitioner of the healing arts toward maintenance, improvement or protection of health or lessening of illness, disability or pain. (US Centers for Medicare & Medicaid Services).

As used herein, the term “Prescription Drug” may refer to The Federal Food Drug and Cosmetic Act (FD&C Act) and FDA regulations which define the term drug, in part, by reference to its intended use, as “articles intended for use in the diagnosis, cure, mitigation, treatment, or prevention of disease” and “articles (other than food) intended to affect the structure or any function of the body of man or other animals.” Therefore, almost any ingested or topical or injectable product that, through its label or labeling (including internet websites, promotional pamphlets, and other marketing material), is claimed to be beneficial for such uses will be regulated by FDA as a drug. The definition also includes components of drugs, such as active pharmaceutical ingredients.

As used herein, the term “Drug Compounding” may refer to the preparation, mixing, assembling, altering, packaging and labelling of a drug, drug-delivery device or device in accordance with a license practitioner's prescription, medication order, or initiative based on the practitioner/patient/pharmacist/compounder relationship in the course of professional practice. This may include customizations which may include: Different Form or Method of Delivery; Custom Strength or Dose; Combine Medications; Allergies or Intolerance to Components of FDA-Approved Drugs; Medications Not Available Commercially; Bioidentical Hormone Replacement; Flavoring

As used herein, the term “Veterinary Prescription Drugs” may refer to Veterinary prescription drugs are those drugs restricted by federal law to use by or on the order of a licensed veterinarian for use in an animal [Section 503(f) Food, Drug, and Cosmetic Act]. The law requires that the drug sponsor label such drugs with the statement: “Caution: Federal law restricts this drug to use by or on the order of a licensed veterinarian.” Veterinary prescription drugs are labeled for use only by or on the order of a licensed veterinarian. Veterinarians making treatment decisions must use sound clinical judgment and current medical information and must be in compliance with federal, state, and local laws and regulations. Veterinary prescription drugs must be properly labeled before being dispensed. Appropriate dispensing and treatment records must be maintained. Veterinary prescription drugs should be dispensed only in quantities required for the treatment of the animal(s) for which the drugs are dispensed. Avoid unlimited refills of prescriptions or any other activity that might result in misuse of drugs. Any drug used in a manner not in accordance with its labeling should be subjected to the same supervisory precautions that apply to veterinary prescription drugs. Orders issued by licensed veterinarians authorize drug distributors to deliver veterinary prescription drugs to a specific client, or authorize pharmacists to dispense such drugs to a specific client.

As used herein, the term “Specialty Pharmacy” may refer to a specialty pharmacy is a state-licensed pharmacy that solely or largely provides only medications for people with serious health conditions requiring complex therapies. These include conditions such as cancer, hepatitis C, rheumatoid arthritis, HIV/AIDS, multiple sclerosis, cystic fibrosis, organ transplantation, human growth hormone deficiencies, and hemophilia and other bleeding disorders. In addition to being state-licensed and regulated, specialty pharmacies are typically accredited by independent third parties such as URAC®, the Accreditation Commission for Health Care (ACHC), the Center for Pharmacy Practice Accreditation (CPPA) or the Joint Commission, in order to ensure consistent quality of care.

Specialty medications have a complex profile that require intensive patient management. Some specialty medications also require special handling. Though some are taken orally, many of these medications need to be injected or infused, some in a doctor's office or hospital. Specialty pharmacies provide services that include training in how to use these medications, comprehensive treatment assessment, patient monitoring, and frequent communication with caregivers and the patient's physician or other healthcare providers. The expert services that specialty pharmacies provide drive adherence and persistency, proper management of medication dosing and side effects, and ensure appropriate medication use.

Specialty drugs are more complex than most prescription medications and are used to treat patients with serious and often life threatening conditions including cancer, hepatitis C, rheumatoid arthritis, HIV/AIDS, multiple sclerosis, cystic fibrosis, organ transplantation, human growth hormone deficiencies, hemophilia and other bleeding disorders. The complexity of these medications may be due to the drug itself, the way it is administered, the management of its side effect profile, the disease or condition it is used to treat, special access conditions required by the manufacturer, payer authorization or benefit requirements, patient financial hardship or any combination of these.

As used herein, the term “Medical Cannabis” may refer to any type of cannabis, although cannabis remains federally illegal in the United States, many states have legalized cannabis for valid medical purposes (and several states have legalized cannabis both medically and for adult use). In order to qualify for medical marijuana, patients must have a diagnosed ailment that is on their state's list of qualifying medical marijuana conditions. With the recommendation of a local physician, a qualified patient can obtain a medical marijuana card or authorization to visit dispensaries and purchase medical marijuana products. (In states where recreational cannabis has been legalized, adult consumers do not need a medical marijuana card, but may not have access to the same medical cannabis products that are available for patients.)

Medical cannabis refers to using the whole cannabis plant, or the plant's basic extracts, for the treatment of various ailments or conditions. The characteristic that defines marijuana from hemp is the content of tetrahydrocannabinol (THC), the compound in cannabis that gets users “high.” Hemp is almost entirely devoid of THC but often high in another cannabinoid—cannabidiol (CBD). Hemp has 0.3 percent THC or less while the threshold for marijuana starts at a THC concentration of 0.31 percent or higher. Both forms of cannabis, hemp and marijuana, have been shown to contain medically beneficial levels of differing cannabinoids, active compounds found in the cannabis plant.

Medical cannabis encompasses synthetic and non-synthetic cannabinoids.

Non-Synthetic cannabis contains over 120 cannabinoids, some of which have been found to have therapeutically beneficial properties. The two major cannabinoids found in cannabis that academic and scientific studies demonstrate to possess the most therapeutic properties are cannabidiol (CBD) and tetrahydrocannabinol (THC), though a number of other cannabinoids, like cannabigerol (CBG) and cannabinol (CBN), also exhibit health benefits.

These cannabinoids interact directly with the body's endocannabinoid system—a signaling network found within every mammalian species on Earth. It features two cannabinoid receptors, CB1 and CB2 receptors, which THC and CBD “dock” with to provide their therapeutic effects. THC, the mind-altering ingredient in cannabis, has been shown to increase appetite, reduce muscle control problems, and reduce nausea, pain, and inflammation. CBD doesn't cause a psychoactive effect like THC, but it has been shown to reduce pain and inflammation, as well as be effective in killing certain cancer cells, controlling epileptic seizures, and treating mental illness.

As used herein, the term “Clinical Laboratory Tests” may refer to a medical procedure that involves testing a sample of blood, urine, or other substance from the body. Laboratory tests can help determine a diagnosis, plan treatment, check to see if treatment is working, or monitor the disease over time.

A clinical (medical) laboratory is a facility that performs testing on materials derived from the human body for the purpose of providing information for the diagnosis, prevention, or treatment of any disease or impairment of, or assessment of the health of, human beings.

Coding for clinical laboratory tests include NCD (National Coverage Determination), ICD-10-CM, HCPCS and CPT codes.

As used herein, the term “Medical Device' may be defined within the Food Drug & Cosmetic Act as an instrument, apparatus, implement, machine, contrivance, implant, in vitro reagent, or other similar or related article, including a component part, or accessory which is: recognized in the official National Formulary, or the United States Pharmacopoeia, or any supplement to them, intended for use in the diagnosis of disease or other conditions, or in the cure, mitigation, treatment, or prevention of disease, in man or other animals, or intended to affect the structure or any function of the body of man or other animals, and which does not achieve any of its primary intended purposes through chemical action within or on the body of man or other animals and which is not dependent upon being metabolized for the achievement of any of its primary intended purposes.

Coding for medical devices includes ICD-10-PCS, CPT and HCPCS

As used herein, the term “Digital Health” may refer to the broad scope of digital health includes categories such as mobile health medical apps (mHealth), health information technology (IT), wireless medical devices, wearable medical devices, telehealth and telemedicine, software as a Medical Device (SaMD) and personalized medicine.

As used herein, the term “computer” refers to a machine, apparatus, or device that is capable of accepting and performing logic operations from software code. The term “application”, “software”, “software code” or “computer software” refers to any set of instructions operable to cause a computer to perform an operation. Software code may be operated on by a “rules engine” or processor. Thus, the methods and systems of the present invention may be performed by a computer or computing device having a processor based on instructions received by computer applications and software.

The term “electronic device” as used herein is a type of computer comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of electronic devices include: personal computers (PCs), workstations, laptops, tablet PCs including the iPad, cell phones including iOS phones made by Apple Inc., Android OS phones, Microsoft OS phones, Blackberry phones, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some non-limiting examples of portable devices include: cell phones, smartphones, tablet computers, laptop computers, wearable computers such as Apple Watch, other smartwatches, Fitbit, other wearable fitness trackers, Google Glasses, and the like.

The term “client device” as used herein is a type of computer or computing device comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of client devices include: personal computers (PCs), workstations, laptops, tablet PCs including the iPad, cell phones including iOS phones made by Apple Inc., Android OS phones, Microsoft OS phones, Blackberry phones, Apple iPads, Anota digital pens, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some non-limiting examples of portable devices include: cell phones, smartphones, tablet computers, laptop computers, tablets, digital pens, wearable computers such as Apple Watch, other smartwatches, Fitbit, other wearable fitness trackers, Google Glasses, and the like.

The term “computer readable medium” as used herein refers to any medium that participates in providing instructions to the processor for execution. A computer readable medium may take many forms, including but not limited to, non-volatile media, volatile media, and transmission media. Non-volatile media includes, for example, optical, magnetic disks, and magneto-optical disks, such as the hard disk or the removable media drive. Volatile media includes dynamic memory, such as the main memory. Transmission media includes coaxial cables, copper wire and fiber optics, including the wires that make up the bus. Transmission media may also take the form of acoustic or light waves, such as those generated during radio wave and infrared data communications.

As used herein the term “data network” or “network” shall mean an infrastructure capable of connecting two or more computers such as client devices either using wires or wirelessly allowing them to transmit and receive data. Non-limiting examples of data networks may include the internet or wireless networks or (i.e. a “wireless network”) which may include Wifi and cellular networks. For example, a network may include a local area network (LAN), a wide area network (WAN) (e.g., the Internet), a mobile relay network, a metropolitan area network (MAN), an ad hoc network, a telephone network (e.g., a Public Switched Telephone Network (PSTN)), a cellular network, a Zigby network, or a voice-over-IP (VoIP) network.

As used herein, the term “database” shall generally mean a digital collection of data or information. The present invention uses novel methods and processes to store, link, and modify information such digital images and videos and user profile information. For the purposes of the present disclosure, a database may be stored on a remote server and accessed by a client device through the internet (i.e., the database is in the cloud) or alternatively in some embodiments the database may be stored on the client device or remote computer itself (i.e., local storage). A “data store” as used herein may contain or comprise a database (i.e. information and data from a database may be recorded into a medium on a data store).

As used herein, the term “blockchain” shall generally mean a distributed database that maintains a continuously growing ledger or list of records, called blocks, secured from tampering and revision using hashes. Every time data may be published to a blockchain database the data may be published as a new block. Each block may include a timestamp and a link to a previous block. Through the use of a peer-to-peer network and a distributed timestamping server, a blockchain database is managed autonomously. Blockchains are an open, distributed ledger that can record transactions between two parties efficiently and in a verifiable and permanent way. Consensus ensures that the shared ledgers are exact copies, and lowers the risk of fraudulent transactions, because tampering would have to occur across many places at exactly the same time. Cryptographic hashes, such as the SHA256 computational algorithm, ensure that any alteration to transaction input results in a different hash value being computed, which indicates potentially compromised transaction input. Digital signatures ensure that transactions originated from senders (signed with private keys) and not imposters. This covers different approaches to the processing including hash trees and hash graphs. At its core, a blockchain system records the chronological order of transactions with all nodes agreeing to the validity of transactions using the chosen consensus model. The result is transactions that are irreversible and agreed to by all members in the network.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

New computer-implemented systems and methods for providing cost effective healthcare that is individually personalized. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention, and is not intended to limit the invention to the specific embodiments illustrated by the figures or description below.

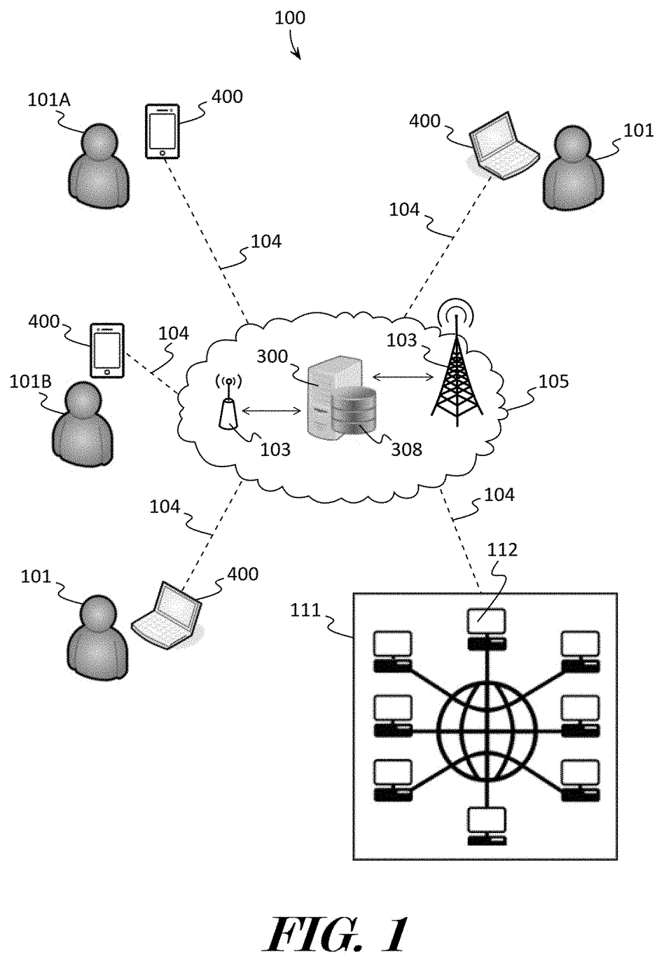

The present invention will now be described by example and through referencing the appended figures representing preferred and alternative embodiments. As perhaps best shown by FIG. 1, an illustrative example of some of the physical components which may comprise a system for predicting the health and therapeutic behavior of individuals (“the system”) 100 according to some embodiments is presented. The system 100 is configured to facilitate the transfer of data and information between one or more access points 103, client devices 400, and servers 300 over a data network 105. Each client device 400 may send data to and receive data from the data network 105 through a network connection 104 with an access point 103. A data store 308 accessible by the server 300 may contain one or more databases. The data may comprise any type of information pertinent to one or more users 101. In preferred embodiments, the data may include healthcare information, such as information on or describing one or more users 101, information on or describing one or more medical conditions 121, information on or describing one or more medications and other therapies used to treat medical conditions 121, information on or describing the cost of one or more medications and other therapies used to treat medical conditions 121, information requested by one or more users 101, information supplied by one or more users 101, and any other information which may be used to provide cost effective healthcare that is individually personalized.

In this example, the system 100 comprises at least one client device 400 (but preferably more than two client devices 400) configured to be operated by one or more users 101. Client devices 400 can be mobile devices, such as laptops, tablet computers, personal digital assistants, smart phones, and the like, that are equipped with a wireless network interface capable of sending data to one or more servers 300 with access to one or more data stores 308 over a network 105 such as a wireless local area network (WLAN). Additionally, client devices 400 can be fixed devices, such as desktops, workstations, and the like, that are equipped with a wireless or wired network interface capable of sending data to one or more servers 300 with access to one or more data stores 308 over a wireless or wired local area network 105. The present invention may be implemented on at least one client device 400 and/or server 300 programmed to perform one or more of the steps described herein. In some embodiments, more than one client device 400 and/or server 300 may be used, with each being programmed to carry out one or more steps of a method or process described herein.

In some embodiments, the system 100 may be configured to facilitate the communication of information to and from one or more users 101, through their respective client devices 400, and servers 300 of the system 100. Users 101 of the system 100 may include one or more healthcare providers (“providers”) 101A and healthcare patients (“patients”) 101B. A provider 101A may include a person, company, or other entity which may provide, authorize, or pay for healthcare services and therapies for a patient 101A. Example providers 101A may include, pharmacies, pharmacists, health insurance companies, doctors, nurses, physician's assistants, anesthesiologists, other specialists, hospitals, clinics, other healthcare entities, and the like. A patient 101B may include a person that has or will receive healthcare services and one or more therapies for a health condition 121, optionally under the care of an authorized agent, such as a family member or guardian.

In some embodiments, the system 100 may include a blockchain network 111, having one or more nodes 112, which may be in communication with one or more servers 300 and/or client devices 400 of the system 100. A node 112 may be a server 300, a client device 400, or any other suitable networked computing platform. The blockchain network 111 may manage a distributed blockchain database 113 containing healthcare information of the system 100. The healthcare information may be maintained as a continuously growing ledger or listing of the data which may be referred to as blocks, secured from tampering and revision. Each block includes a timestamp and a link to a previous block. Through the use of a peer-to-peer blockchain network 111 and a distributed timestamping server 300, a blockchain database 113 may be managed autonomously. Consensus ensures that the shared ledgers are exact copies, and lowers the risk of fraudulent transactions, because tampering would have to occur across many places at exactly the same time. Cryptographic hashes, such as the SHA256 computational algorithm, ensure that any alteration to transaction data input results in a different hash value being computed, which indicates potentially compromised transaction input. Digital signatures ensure that data entry transactions (data added to the blockchain database 113) originated from senders (signed with private keys) and not imposters. At its core, a blockchain database 113 may record the chronological order of data entry transactions with all nodes 112 agreeing to the validity of entry transactions using the chosen consensus model. The result is data entry transactions that are irreversible and agreed to by all members in the blockchain network 111.

The blockchain network 111 may comprise a cryptocurrency or digital asset designed to work as a medium of exchange that uses cryptography to secure its transactions, to control the creation of additional units, and to verify the transfer of assets. Example cryptocurrencies include Bitcoin, Ethereum, Ripple, etc. The blockchain network 111 may also comprise tokens 132 common to cryptocurrency based blockchain networks 111. The tokens 132 may serve as a reward or incentive to nodes 112 for blockchain network 111 services and to make the blockchain network 111 attach resistant. The blockchain network 111 may comprise token governance rulesets based on crypto economic incentive mechanisms that determine under which circumstances blockchain network 111 transactions are validated and new blocks are created. Tokens 132 may include usage tokens, work tokens, Intrinsic, Native or Built-in tokens, application token, asset-backed tokens, or any other type of token which may be used in a cryptocurrency network.

In preferred embodiments, the system 100 and methods disclosed herein may use the blockchain database 113 of the blockchain network 111 to enable a novel pharmacy benefits management healthcare model, preferably through creating Smart Healthcare contracts, to predict medication usage and spending of patients 101B over time. In further embodiments, patients 101B may benefit as tokenized/cryptocurrency is available for anonymized data collection and data maintenance which may eliminate copayments and coinsurances due from the patient 101B, such that the healthcare data 120 of one or more patients 101B may be associated with cryptocurrency token(s) 132 of the system 100. In still further embodiments, shared anonymized data of the system may be purchased by healthcare providers 101A, such as pharma and health plan providers, to undergird the cryptocurrency coin/token 132 value. In preferred embodiments, all or portions of one or more third-party cryptocurrency tokens 132 may be used as payment for a patient 101B to receive one or more therapies, such as a possible therapy 127.

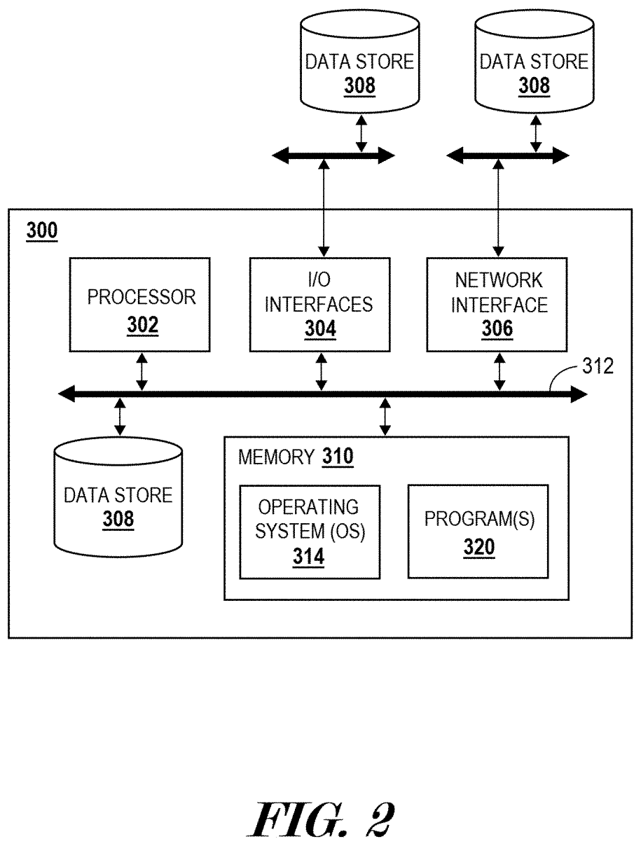

Referring now to FIG. 2, in an exemplary embodiment, a block diagram illustrates a server 300 of which one or more may be used in the system 100 or standalone and which may be a type of computing platform. The server 300 may be a digital computer that, in terms of hardware architecture, generally includes a processor 302, input/output (I/O) interfaces 304, a network interface 306, a data store 308, and memory 310. It should be appreciated by those of ordinary skill in the art that FIG. 2 depicts the server 300 in an oversimplified manner, and a practical embodiment may include additional components and suitably configured processing logic to support known or conventional operating features that are not described in detail herein. The components (302, 304, 306, 308, and 310) are communicatively coupled via a local interface 312. The local interface 312 may be, for example but not limited to, one or more buses or other wired or wireless connections, as is known in the art. The local interface 312 may have additional elements, which are omitted for simplicity, such as controllers, buffers (caches), drivers, repeaters, and receivers, among many others, to enable communications. Further, the local interface 312 may include address, control, and/or data connections to enable appropriate communications among the aforementioned components.

The processor 302 is a hardware device for executing software instructions. The processor 302 may be any custom made or commercially available processor, a central processing unit (CPU), an auxiliary processor among several processors associated with the server 300, a semiconductor-based microprocessor (in the form of a microchip or chip set), or generally any device for executing software instructions. When the server 300 is in operation, the processor 302 is configured to execute software stored within the memory 310, to communicate data to and from the memory 310, and to generally control operations of the server 300 pursuant to the software instructions. The I/O interfaces 304 may be used to receive user input from and/or for providing system output to one or more devices or components. User input may be provided via, for example, a keyboard, touch pad, and/or a mouse. System output may be provided via a display device and a printer (not shown). I/O interfaces 304 may include, for example, a serial port, a parallel port, a small computer system interface (SCSI), a serial ATA (SATA), a fibre channel, Infiniband, iSCSI, a PCI Express interface (PCI-x), an infrared (IR) interface, a radio frequency (RF) interface, and/or a universal serial bus (USB) interface.

The network interface 306 may be used to enable the server 300 to communicate on a network, such as the Internet, the data network 105, the enterprise, and the like, etc. The network interface 306 may include, for example, an Ethernet card or adapter (e.g., 10 BaseT, Fast Ethernet, Gigabit Ethernet, 10 GbE) or a wireless local area network (WLAN) card or adapter (e.g., 802.11a/b/g/n). The network interface 306 may include address, control, and/or data connections to enable appropriate communications on the network. A data store 308 may be used to store data.

The data store 308 is a type of memory and may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 308 may incorporate electronic, magnetic, optical, and/or other types of storage media. In one example, the data store 308 may be located internal to the server 300 such as, for example, an internal hard drive connected to the local interface 312 in the server 300. Additionally in another embodiment, the data store 308 may be located external to the server 300 such as, for example, an external hard drive connected to the I/O interfaces 304 (e.g., SCSI or USB connection). In a further embodiment, the data store 308 may be connected to the server 300 through a network, such as, for example, a network attached file server.

The memory 310 may include any of volatile memory elements (e.g., random access memory (RAM, such as DRAM, SRAM, SDRAM, etc.)), nonvolatile memory elements (e.g., ROM, hard drive, tape, CDROM, etc.), and combinations thereof. Moreover, the memory 310 may incorporate electronic, magnetic, optical, and/or other types of storage media. Note that the memory 310 may have a distributed architecture, where various components are situated remotely from one another, but can be accessed by the processor 302. The software in memory 310 may include one or more software programs, each of which includes an ordered listing of executable instructions for implementing logical functions. The software in the memory 310 may include a suitable operating system (O/S) 314 and one or more programs 320.

The operating system 314 essentially controls the execution of other computer programs, such as the one or more programs 320, and provides scheduling, input-output control, file and data management, memory management, and communication control and related services. The operating system 314 may be, for example Windows NT, Windows 2000, Windows XP, Windows Vista, Windows 7, Windows 8, Windows 10, Windows Server 2003/2008 (all available from Microsoft, Corp. of Redmond, Wash.), Solaris (available from Sun Microsystems, Inc. of Palo Alto, Calif.), LINUX (or another UNIX variant) (available from Red Hat of Raleigh, N.C. and various other vendors), Android and variants thereof (available from Google, Inc. of Mountain View, Calif.), Apple OS X and variants thereof (available from Apple, Inc. of Cupertino, Calif.), or the like.

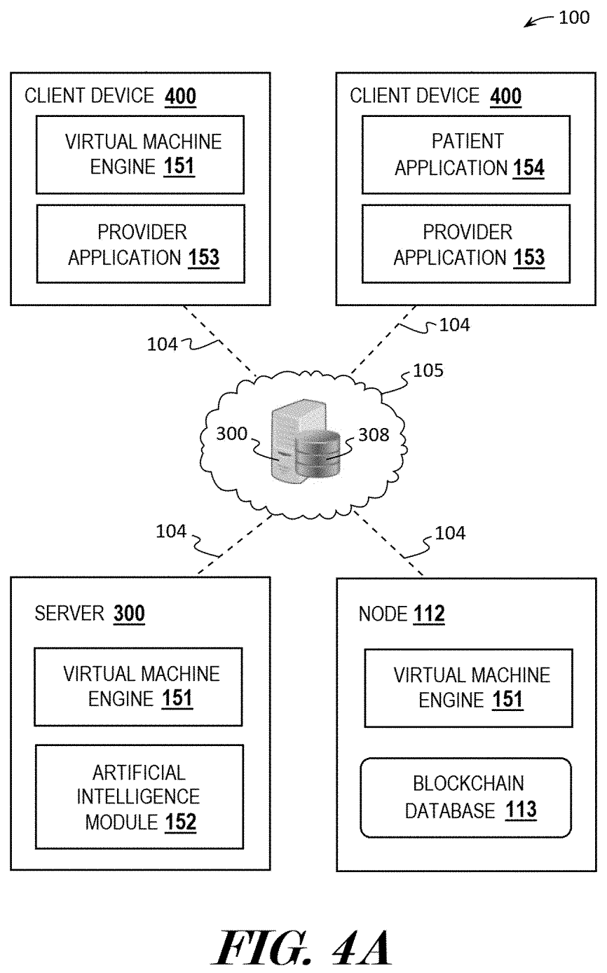

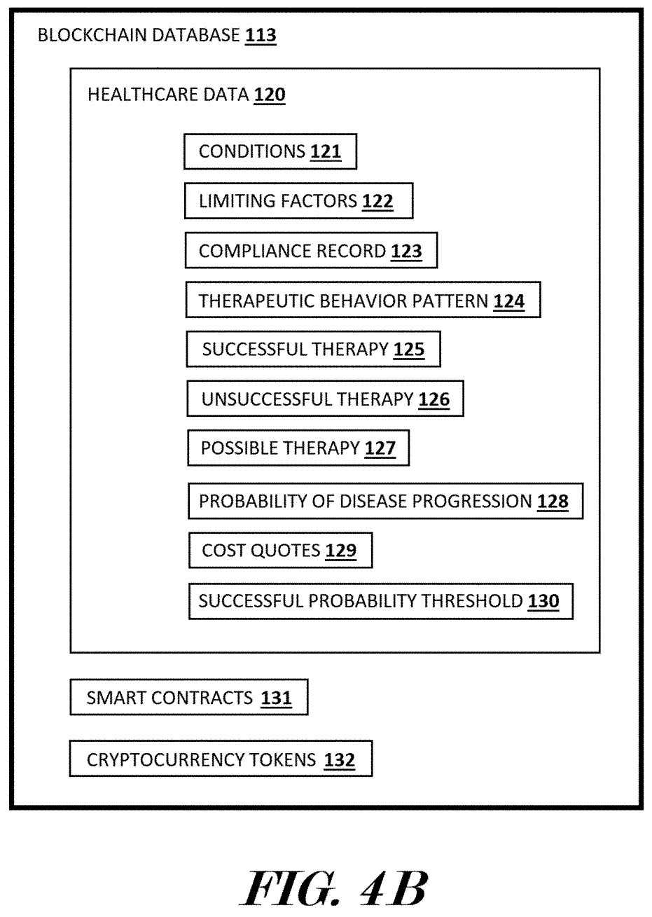

The one or more programs 320 may include a virtual machine engine 151 (FIG. 4A), an artificial intelligence module 152 (FIG. 4A), and a provider application 153 (FIG. 4A), and the programs 320 may be configured to implement the various processes, algorithms, methods, techniques, etc. described herein.

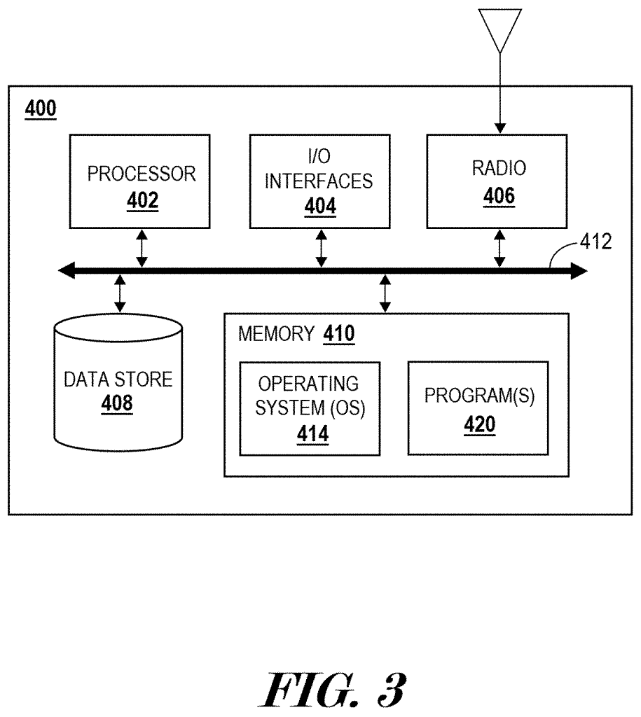

Referring to FIG. 3, in an exemplary embodiment, a block diagram illustrates a client device 400 of which one or more may be used in the system 100 or the like and which may be a type of computing platform. The client device 400 can be a digital device that, in terms of hardware architecture, generally includes a processor 402, input/output (I/O) interfaces 404, a radio 406, a data store 408, and memory 410. It should be appreciated by those of ordinary skill in the art that FIG. 3 depicts the client device 400 in an oversimplified manner, and a practical embodiment may include additional components and suitably configured processing logic to support known or conventional operating features that are not described in detail herein. The components (402, 404, 406, 408, and 410) are communicatively coupled via a local interface 412. The local interface 412 can be, for example but not limited to, one or more buses or other wired or wireless connections, as is known in the art. The local interface 412 can have additional elements, which are omitted for simplicity, such as controllers, buffers (caches), drivers, repeaters, and receivers, among many others, to enable communications. Further, the local interface 412 may include address, control, and/or data connections to enable appropriate communications among the aforementioned components.

The processor 402 is a hardware device for executing software instructions. The processor 402 can be any custom made or commercially available processor, a central processing unit (CPU), an auxiliary processor among several processors associated with the client device 400, a semiconductor-based microprocessor (in the form of a microchip or chip set), or generally any device for executing software instructions. When the client device 400 is in operation, the processor 402 is configured to execute software stored within the memory 410, to communicate data to and from the memory 410, and to generally control operations of the client device 400 pursuant to the software instructions. In an exemplary embodiment, the processor 402 may include a mobile optimized processor such as optimized for power consumption and mobile applications.

The I/O interfaces 404 can be used to receive data and user input and/or for providing system output. User input can be provided via a plurality of I/O interfaces 404, such as a keypad, a touch screen, a camera, a microphone, a scroll ball, a scroll bar, buttons, bar code scanner, voice recognition, eye gesture, and the like. System output can be provided via a display screen 404A such as a liquid crystal display (LCD), touch screen, and the like. The I/O interfaces 404 can also include, for example, a global positioning service (GPS) radio, a serial port, a parallel port, a small computer system interface (SCSI), an infrared (IR) interface, a radio frequency (RF) interface, a universal serial bus (USB) interface, and the like. The I/O interfaces 404 can include a graphical user interface (GUI) that enables a user to interact with the client device 400. Additionally, the I/O interfaces 404 may be used to output notifications to a user and can include a speaker or other sound emitting device configured to emit audio notifications, a vibrational device configured to vibrate, shake, or produce any other series of rapid and repeated movements to produce haptic notifications, and/or a light emitting diode (LED) or other light emitting element which may be configured to illuminate to provide a visual notification.

The radio 406 enables wireless communication to an external access device or network. Any number of suitable wireless data communication protocols, techniques, or methodologies can be supported by the radio 406, including, without limitation: RF; IrDA (infrared); Bluetooth; ZigBee (and other variants of the IEEE 802.15 protocol); IEEE 802.11 (any variation); IEEE 802.16 (WiMAX or any other variation); Direct Sequence Spread Spectrum; Frequency Hopping Spread Spectrum; Long Term Evolution (LTE); cellular/wireless/cordless telecommunication protocols (e.g. 3G/4G, etc.); wireless home network communication protocols; paging network protocols; magnetic induction; satellite data communication protocols; wireless hospital or health care facility network protocols such as those operating in the WMTS bands; GPRS; proprietary wireless data communication protocols such as variants of Wireless USB; and any other protocols for wireless communication.

The data store 408 may be used to store data and is therefore a type of memory. The data store 408 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 408 may incorporate electronic, magnetic, optical, and/or other types of storage media.

The memory 410 may include any of volatile memory elements (e.g., random access memory (RAM, such as DRAM, SRAM, SDRAM, etc.)), nonvolatile memory elements (e.g., ROM, hard drive, etc.), and combinations thereof. Moreover, the memory 410 may incorporate electronic, magnetic, optical, and/or other types of storage media. Note that the memory 410 may have a distributed architecture, where various components are situated remotely from one another, but can be accessed by the processor 402. The software in memory 410 can include one or more software programs 420, each of which includes an ordered listing of executable instructions for implementing logical functions. In the example of FIG. 3, the software in the memory system 410 includes a suitable operating system (O/S) 414 and programs 420.

The operating system 414 essentially controls the execution of other computer programs, and provides scheduling, input-output control, file and data management, memory management, and communication control and related services. The operating system 414 may be, for example, LINUX (or another UNIX variant), Android (available from Google), Symbian OS, Microsoft Windows CE, Microsoft Windows 7 Mobile, Microsoft Windows 10, iOS (available from Apple, Inc.), webOS (available from Hewlett Packard), Blackberry OS (Available from Research in Motion), and the like.

The programs 420 of a client device 400 may include a virtual machine engine 151 (FIG. 4A), a provider application 153 (FIG. 4A), a patient application 154 (FIG. 4A), and various applications, add-ons, etc. configured to provide end user functionality with the client device 400. For example, exemplary programs 420 may include, but not limited to, a web browser, social networking applications, streaming media applications, games, mapping and location applications, electronic mail applications, financial applications, and the like. In a typical example, the end user typically uses one or more of the programs 420 along with a network 105 to manipulate information of the system 100.

Referring now to FIG. 4A, a block diagram showing some software rules engines and databases which may be found in a system 100 is illustrated. In some embodiments, the system 100 may comprise a virtual machine engine 151, an artificial intelligence module 152, a provider application 153, and/or a patient application 154. The engines 151, 152, 153, 154, may comprise one or more of the programs 320 of a server 300, and/or programs 420 of a client device 400 of the system 100. Preferably, the system 100 may comprise a blockchain network 111 comprising one or more nodes 112 in which one or more servers 300 and client devices 400 may function as or comprise one or more of the nodes 112. Each node 112 of the blockchain network 111 may maintain a blockchain database 113 which may comprise a distributed ledger of the blockchain network 111. One or more of the engines 151, 152, 153, 154, may read, write, or otherwise access data in the blockchain database(s) 113 of the system 100. Additionally, the engines 151, 152, 153, 154, may be in electronic communication so that data may be readily exchanged between the engines 151, 152, 153, 154. It should be understood that the functions attributed to the engines 151, 152, 153, 154, described herein are exemplary in nature, and that in alternative embodiments, any function attributed to any engine 151, 152, 153, 154, may be performed by one or more other engines 151, 152, 153, 154, or any other suitable processor logic.

A virtual machine engine 151 may comprise or function as virtual machine logic stored in memory 310, 410 which may be executable by the processor 302, 402, of one or more servers 300 and/or client devices 400 that may be functioning as a node 112. In some embodiments, the virtual machine engine 151 may manage and perform data transactions on the blockchain database 113 of a node 112, and the virtual machine engine 151 may be run by a processor 302, 402, of a node 112, to maintain a distributed ledger (copy of the blockchain database 113) on its memory 310, 410, and may thus synchronize transaction data with other nodes 112 containing the distributed ledger in order to implement a blockchain based transaction processing system 100. In further embodiments, a virtual machine engine 151 may provide access to data of the blockchain database 113 of a server 300 or client device 400.

As shown in FIG. 4B, the system 100 may comprise one or more blockchain databases 113 that may contain healthcare data, which may include healthcare data of one or more, and preferably a plurality of, patients 101B. A blockchain database 113 may comprise a distributed ledger in which a copy of the blockchain database 113 is stored and maintained by one or more nodes 112 of a blockchain network 111. In some embodiments, the data of a blockchain databases 113 may be maintained as a continuously growing ledger or listing of the data, which may be referred to as blocks, secured from tampering and revision. Each block includes a timestamp and a link to a previous block. Through the use of a peer-to-peer blockchain network 111 and a distributed timestamping server 300, a blockchain database 113 may be managed autonomously. Consensus ensures that the shared ledgers are exact copies, and lowers the risk of fraudulent transactions, because tampering would have to occur across many places at exactly the same time. Cryptographic hashes, such as the SHA256 computational algorithm, ensure that any alteration to transaction data input results in a different hash value being computed, which indicates potentially compromised transaction input. Digital signatures ensure that data entry transactions (data added to the blockchain database 1113) originated from senders (signed with private keys) and not imposters. At its core, a blockchain database 113 may record the chronological order of data entry transactions with all nodes 112 agreeing to the validity of entry transactions using the chosen consensus model. The result is data entry transactions that are irreversible and agreed to by all members in the blockchain network 111.

In some embodiments, a blockchain database 113 may store the healthcare data 120 of a plurality of patients 101B. Preferably, the healthcare data 120 of each patient 101B may be encrypted in the blockchain database 113. In further embodiments, the healthcare data 120 of a patient 101B may include one or more conditions 121, limiting factors 122, compliance records 123, therapeutic behavior patterns 124, successful therapies 125, unsuccessful therapies 126, possible therapies 127, probability of disease progression 128, cost quotes 129, and successful probability thresholds 130. In still further embodiments, a blockchain database 113 may store one or more smart contracts 131 and cryptocurrency tokens 132 which may be associated with the healthcare data 120 of one or more patients 101B.

An artificial intelligence module 152 may comprise or function as artificial intelligence logic stored in memory 310, 410 which may be executable by the processor 302, 402, of one or more servers 300 and/or client devices 400. In some embodiments, the artificial intelligence module 152 may function as or comprise a machine/deep learning/artificial intelligence platform that interrogates the healthcare information or data of the system 100 and learns about healthcare behaviors and trends of one or more patients 101B. In further embodiments, the artificial intelligence module 152 may function to provide and recommend solutions, such as therapies which are cost effective and which may successfully treat a condition 121 of a patient 101B, to patients and healthcare providers 101A. In still further embodiments, the artificial intelligence module 152 may function to generate population data and other informatics, such as anonymized general patient population data, for healthcare organizations and Pharma using information of one or more patients 101B stored in one or more data stores 308, 408, and/or blockchain databases 113.

A provider application 153 may comprise or function as provider logic stored in memory 310, 410 which may be executable by the processor 302, 402, of one or more servers 300 and/or client devices 400. In some embodiments, a provider application 153 may provide a user interface, such as a dashboard, that allows providers 101A and healthcare organizations to review patient 101B consented details and healthcare information. In further embodiments, a provider application 153 may function to provide or effect payment for healthcare services.

A patient application 154 may comprise or function as patient logic stored in memory 310, 410 which may be executable by the processor 302, 402, of one or more servers 300 and/or client devices 400. In some embodiments, a patient application 154 may provide a user interface, such as a portal, that allows patients 101B to review their medical needs in one convenient application to identify appropriate or successful therapies 125 and treatments. In further embodiments, a patient application 154 may provide a user interface that allows patients 101B to gain or access financial incentives through following or complying with treatments and therapies, improving their health, and sharing analyzed data with third party organizations as well as paying for drugs via cryptocurrency of the system 100 or other payment option. In still further embodiments, a patient application 154 may provide a user interface that allows a prescriber or other healthcare provider 101A to receive incentives, which may include cryptocurrency, other financial incentives, goods, services, or any other type of incentive, for working with the system 100 to keep a patient 101B on appropriate cost-effective therapies.

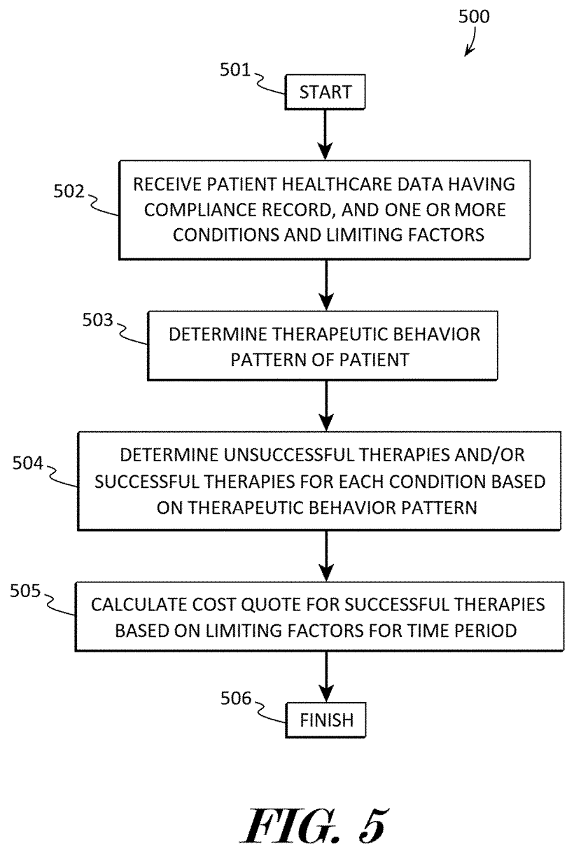

FIG. 5 shows a block diagram of an example of a computer-implemented method for predicting the health and therapeutic behavior of patients (“the method”) 500 according to various embodiments described herein. In some embodiments, the method 500 may be used to predict changes in a patient 101B as well as provide information which may be used to predict a healthier lifestyle for the patient 101B through changes in lifestyle circumstances, changes in drug types and information on interactions with other drugs that the patient 101B is taking. Furthermore, the method 500 may be used to predict drug usage for a patient 101B and to outline a drug usage and cost schedule for a period of time. One or more steps of the method 500 may be performed by a virtual machine engine 151, artificial intelligence module 152, provider application 153, and/or patient application 154 which may be executed by a computing device processor, such as a processor 302 (FIG. 2) and/or a processor 402 (FIG. 3).

The method 500 may start 501 and healthcare data of a patient 101B having a compliance record 123, one or more conditions 121 and limiting factors 122 may be received in step 502. In some embodiments, the healthcare data may be received by an artificial intelligence module 152 from the client device 400 of a provider 101A and/or patient 101B. In further embodiments, the healthcare data may be retrieved from the blockchain database 113 via a virtual machine engine 151 preferably of a client device 400. Limiting factors 122 of a patient 101B may include existing drugs and therapies, lifestyle behaviors (such as smoking, weight changes, and mental health situations), if the patient 101B is ambulatory, or any other information which may limit the ability of the patient 101B to access or complete a therapy. Conditions 121 of a patient 101B may include any type of health condition 121, such as asthma, hypertension, hypercholesteremia, fragile X syndrome, depression, penicillin allergy, and any other condition 121 or disease state which may affect the health and wellbeing of the patient 101B. A compliance record 123 of a patient 101B may include data which describes the dosing schedule for one or more medications prescribed to the patient 101B along with the amount and timing of the refills for the one or more medications that the patient 101B has received. As an example, in step 502, the artificial intelligence module 152 may receive healthcare data of a patient 101B having an existing condition 121 of eczema, a new condition 121 of athletes' foot, a limiting factor 122 of bipolar disorder, and a compliance record 123 for the existing condition 121 that includes refill information on an oral prescription and a topical prescription for the existing condition 121 of eczema.

In step 503, the therapeutic behavior pattern 124 of the patient 101B may be determined. In some embodiments, an artificial intelligence module 152 may use the healthcare information of the patient 101B to review the compliance record 123 of the patient 101B during one or more therapies to determine the therapeutic behavior pattern 124 of the patient 101B. For example, the artificial intelligence module 152 may review prescription refill information to determine if the patient 101B has a history of taking medications correctly or the artificial intelligence module 152 may review if a patient is following a dialysis schedule as directed. By determining the therapeutic behavior pattern 124 of the patient 101B, the artificial intelligence module 152 may determine how likely the patient 101B will be compliant with future therapies. Continuing the above example, a compliance record 123 for a patient 101B may include oral prescriptions and topically applied prescriptions for the existing condition 121 of eczema, with the patient 101B consistently refilling the topically applied prescriptions, but sporadically filling the oral prescriptions. The intelligence module 152 may determine that the therapeutic behavior pattern 124 of the patient 101B has a high probability of compliance for topically applied prescriptions and a low probability of compliance for oral prescriptions.

In step 504, one or more unsuccessful therapies 126 and/or successful therapies 125 for each condition 121 may be determined by the artificial intelligence module 152 based on the therapeutic behavior pattern 124 of step 503. Therapies 125, 126, may include: procedures, such as laboratory tests; medical devices and digital health technologies; prescriptive drugs, such as prescription drugs, compounded drugs, veterinary prescription drugs, specialty pharmacy medications, medical cannabis; phytocannabinoids; terpenoid molecules; other compounds; or any other therapy which may be used to treat a condition 121 for that patient 101B. In some embodiments, after assessment of a current disease state status, the artificial intelligence module 152 may aggregate a review of medications and therapies that have been tried and failed (or those of similar mechanism of action and/or potency) as well as the current medications being taken. From this evaluation the artificial intelligence module 152 may compute and determine precluded or unsuccessful therapies 126 (those that wouldn't be prescribed) and possible successful therapies 125, including pharmacotherapeutic considerations, for that patient 101B. Continuing the above example, the artificial intelligence module 152 may determine that a first therapy of a first topical prescription for the new condition 121 of athletes' foot would not interfere with the patient's 101B current topical prescription for eczema and based on the therapeutic behavior pattern 124 of the patient 101B having a high probability of compliance for topically applied prescriptions, the artificial intelligence module 152 may determine that the first therapy of a first topical prescription for the new condition 121 of athletes' foot would be a successful therapy 125 which may be used to treat the new condition 121. As another example, the artificial intelligence module 152 may determine that a second therapy of a second oral prescription for the new condition 121 of athletes' foot would interfere with the patient's 101B current topical prescription for eczema and based on the therapeutic behavior pattern 124 of the patient 101B having a low probability of compliance for oral prescriptions, the artificial intelligence module 152 may determine that the second oral prescription for the new condition 121 of athletes' foot would be an unsuccessful therapy 126 which may not be used to treat the new condition 121.

In further embodiments, one or more unsuccessful therapies 126 and/or successful therapies 125 for each condition 121 may be determined by the artificial intelligence module 152 based on the one or more limiting factors 122 of step 502. Optionally, after assessment of a current disease state status, the artificial intelligence module 152 may aggregate a review of the one or more limiting factors 122 of the patient 101B. From this evaluation the artificial intelligence module 152 may compute and determine precluded or unsuccessful therapies 126 (those that wouldn't be prescribed) and possible successful therapies 125, including pharmacotherapeutic considerations, for that patient 101B. Continuing the above example, the artificial intelligence module 152 may determine that a third therapy of a third topical prescription for the new condition 121 of athletes' foot would not interfere with the patient's 101B current topical prescription for eczema and that the limiting factor 122 of bipolar disorder has little to no impact on the typical patients' ability to take the third topical prescription as prescribed. The artificial intelligence module 152 may then determine that the third therapy of a third topical prescription for the new condition 121 of athletes' foot would be a successful therapy 125 which may be used to treat the new condition 121. As a further example, the artificial intelligence module 152 may determine that a fourth therapy of a fourth topical prescription for the new condition 121 of athletes' foot would not interfere with the patient's 101B current topical prescription for eczema and that the limiting factor 122 of bipolar disorder has a significant impact on the typical patients' ability to take the third topical prescription as prescribed due to it having a possible side effect of exacerbating bipolar symptoms. The artificial intelligence module 152 may then determine that the fourth therapy of a fourth topical prescription for the new condition 121 of athletes' foot would be an unsuccessful therapy 126 which may not be used to treat the new condition 121.

In step 505, a cost quote 129 for the successful therapies 125 determined in step 504 may be calculated based on the limiting factors 122 for a desired time period. In some embodiments, the artificial intelligence module 152 may calculate the cost of each therapy which may be used to successfully treat a condition 121 of the patient 101B. The cost calculation may include weighting the limiting factors 122 of the patient 101B. For example, a patient 101B that has a limiting factor 122 of a poor record of attending checkups and doctor visits may add a twenty percent increase to the cost of a therapy which requires frequent patient checkups and monitoring. Additionally, the cost calculation may include the cost of providing the therapy to the patient 101B for a desired or specified time period, such as six months or a year. In this manner, a cost quote 129 may be provided by the artificial intelligence module 152 for each possible therapy 127 which may be used to treat a patient 101B for a condition 121. The cost quote 129 may be saved in the healthcare information of the patient 101B stored in the blockchain database 113 which may be made available to the patient 101B via a patient application 154 and to one or more providers 101A via a provider application 153. In preferred embodiments, the fixed fee quote may be made available to payers so that a determination can be made to institute fixed-fee schedule. After step 505, the method 500 may finish 506.

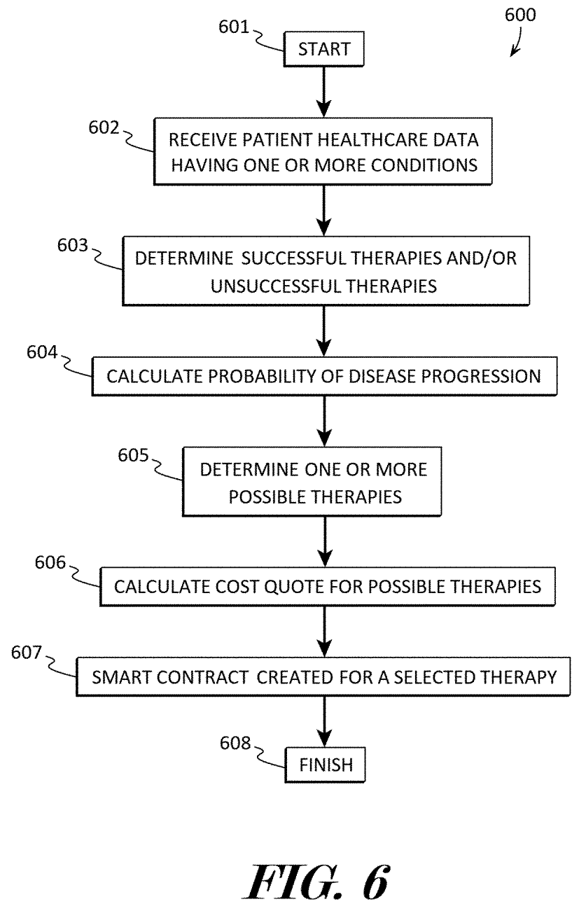

FIG. 6 depicts a block diagram of an example of a computer-implemented method of providing costs effective therapy for a patient (“the method”) 600 according to various embodiments described herein. In some embodiments, the method 600 may be used to predict the cost of providing a therapy to a patient 101B to treat a condition 121 of the patient 101B in which the patient has one or more other conditions 121 that the patient 101B may be being treated for. One or more steps of the method 600 may be performed by a virtual machine engine 151, artificial intelligence module 152, provider application 153, and/or patient application 154 which may be executed by a computing device processor, such as a processor 302 (FIG. 2) and/or a processor 402 (FIG. 3).

The method 600 may start 601 and healthcare data for a patient 101B may be received in step 602. In preferred embodiments, the healthcare data for a patient 101B received in step 602 may include one or more new conditions 121 for the patient 101B. In some embodiments, the healthcare data may be received by an artificial intelligence module 152 from the client device 400 of a provider 101A and/or patient 101B. In further embodiments, the healthcare data may be retrieved from the blockchain database 113 via a virtual machine engine 151 preferably of a client device 400. The patient 101B, provider 101A, and/or payer may provide healthcare data to the system 100 which may include drug data, such as the National Drug Code (NDC), primary coding, secondary coding (preferably via International Classification of Diseases, Tenth Revision, Clinical Modification (ICD-10-CM) coding), co-morbidities, genomics, lifestyle choices, laboratory values, a compliance record 123, one or more conditions 121 and limiting factors 122 which may be stored in a blockchain database 113. In further embodiments, the healthcare data may be retrieved from the blockchain database 113 via a virtual machine engine 151. Limiting factors 122 of a patient 101B may include existing drugs and therapies, lifestyle behaviors (such as smoking, weight changes, and mental health situations), if the patient 101B is ambulatory, or any other information which may limit the ability of the patient 101B to access or complete a therapy.

In step 603 one or more successful therapies 125 and/or unsuccessful therapies 126 may be determined for the one or more new conditions 121 for the patient 101B. Preferably, the artificial intelligence module 152 may determine one or more therapeutic categories along with the unsuccessful therapies 126 and successful therapies 125. In some embodiments, the artificial intelligence module 152 may assess the current disease state status of a condition 121 of the patient 101B, and the artificial intelligence module 152 may aggregate a review of therapies, such as medications, that have been tried and failed (or those of similar mechanism of action and/or potency) as well as the current medications being taken by the patient 101B. From this evaluation the artificial intelligence module 152 may compute and determine precluded or unsuccessful therapies 126 (those that wouldn't be prescribed) and possible successful therapies 125, including pharmacotherapeutic considerations, for that patient 101B. Therapies 125, 126, may include: procedures, such as laboratory tests; medical devices and digital health technologies; prescriptive drugs, such as prescription drugs, compounded drugs, veterinary prescription drugs, specialty pharmacy medications, medical cannabis; phytocannabinoids; terpenoid molecules; other compounds; or any other therapy which may be used to treat a condition 121 for that patient 101B. In further embodiments, all possible therapeutic and pharmacologic combinations of therapies may be examined and determined to be pharmacologically and therapeutically appropriate. In still further embodiments, therapies may be included or precluded based on co-morbidities, drug-drug interactions, drug-disease interactions, drug-laboratory interactions as well as genomic considerations.

In step 604, the probability of disease progression 128 of a new condition 121 of the patient 101B may be calculated. In some embodiments, the artificial intelligence module 152 may perform the calculation for each condition 121 of the patient 101B. For example, the patient 101B may have a primary condition 121, secondary condition 121, tertiary condition 121 of any other number of conditions 121. The artificial intelligence module 152 may determine the unknown variable of the likelihood of disease progression, resolution or stability that queries the categories indicated, that would be indicated, or potentially indicated, but not contraindicated, to treat a primary condition 121 based upon the healthcare information of the blockchain database 113, such as historical therapies that may include prescriptive drugs and/or phytocannabinoids and/or terpenoid compounds or meds taken, the current prescription drugs and/or phytocannabinoid and/or terpenoid compounds or meds taken and the patient's response to them, as well as to the mechanism of action and the potential prescription drugs and/or phytocannabinoid and/or terpenoid agents used for the primary condition 121 to control the primary condition 121, as the response can be as expected, above expected or below expected, therefore the condition 121 may maintain, progress or recede. Historical therapies may also include: procedures, such as laboratory tests; medical devices and digital health technologies; prescriptive drugs, such as prescription drugs, compounded drugs, veterinary prescription drugs, specialty pharmacy medications, medical cannabis; phytocannabinoids; terpenoid molecules; other compounds; or Then the secondary condition 121 or limiting factor 122 if present may preclude some prescriptive and/or phytocannabinoid and/or terpenoid agents, categories based on that condition 121 and the likelihood of progression or resolution based upon the global objective and/or subjective assessment of the condition 121 of the patient 101B over the fixed time period under consideration. The process and its inherent complexity increases with each condition 121 and/or limiting factor 122 presented and may be repeated for a tertiary condition 121 and any other number of conditions 121.

In step 605, one or more possible therapies 127 for a new condition 121 may be determined by the artificial intelligence module 152. Possible therapies 127 may include: procedures, such as laboratory tests; medical devices and digital health technologies; prescriptive drugs, such as prescription drugs, compounded drugs, veterinary prescription drugs, specialty pharmacy medications, medical cannabis; phytocannabinoids; terpenoid molecules; other compounds; or any other therapy which may be used to treat a condition 121 of a patient 101B. The artificial intelligence module 152 may use the successful therapies 125 determined in step 603 as the possible therapies 127 which may be used to treat the new condition 121. Possible therapies 127 may include prescriptive drugs, phytocannabinoids, terpenoid agents, or any other treatment which may be used to treat the condition 121. In preferred embodiments, the possible therapies 127 may be ranked by probability of successful treatment of a new condition 121 of the patient 101B, such as the primary condition 121, may be determined by the artificial intelligence module 152. Preferably, all possible therapies 127 may be determined and may be assigned ranking for successful treatment. The artificial intelligence module 152 may rank the possible therapies 127 based on the likelihood of the therapy successfully treating the condition 121. This likelihood may be based on the compliance record 123 and one or more conditions 121 and limiting factors 122 of the patient 101B. As an example, an artificial intelligence module 152 may determine two possible therapies 127, such as a first therapy and a second therapy, and the artificial intelligence module 152 may rank the first and second possible therapies 127 by probability of the first and second possible therapies 127 being a successful treatment. As a further example, the artificial intelligence module 152 may rank the second possible therapy 127 (an oral phytocannabinoid) higher than the first possible therapy 127 (a topical prescription) since the patient has a higher compliance record 123 with oral therapies than with topical therapies.

In step 606, a cost quote 129 for the possible therapies 127 may be calculated. Preferably, a fixed quote may be provided for each possible therapy 127 using ICD-10 coding and the NDC code for all drug therapies required to successfully and therapeutically treat the patient 101B for fixed period of time may be calculated by the artificial intelligence module 152. In some embodiments, artificial intelligence module 152 may calculate the cost quote 129 for one or more therapies which exceed a successful probability threshold 130. For example, the artificial intelligence module 152 may calculate the cost quote 129 for all therapies having a probability of successfully treating the condition 121 that is greater than seventy fiver percent. The artificial intelligence module 152 may determine the overall function and economics of a fixed period of time with an unknown variable of the likelihood of disease progression. One or more categories of therapies, such as prescription drugs and/or phytocannabinoids and/or terpenoid could be used with or in place of the current prescriptive drugs and/or phytocannabinoid and/or terpenoid regimen considering the best and worst-case scenarios to generate a list of candidate prescriptive drugs and/or cannabinoid and/or terpenoid molecules or compounds relative to the economics, preferably over a fixed period of time. The categories of therapies may also include: procedures, such as laboratory tests; medical devices and digital health technologies; prescriptive drugs, such as prescription drugs, compounded drugs, veterinary prescription drugs, specialty pharmacy medications, medical cannabis; phytocannabinoids; terpenoid molecules; other compounds; or any other therapy which may be used to treat a condition 121 of a patient 101B. These cost quotes 129 and possible therapies 127 may be provided to a patient 101B via a patient application 154 and to a provider 101A via a provider application 153. In some embodiments, each patient subject variable set may be weighted as to the likelihood of occurrence to calculate the potential need for prescriptive and/or phytocannabinoid and/or terpenoid drug change which results impact the cost variable. Patient 101B categories may be disease based, prescriptive and/or phytocannabinoid and/or terpenoid drug based and can be utilized to impact patient contribution or co-payment to affect disease control and improved outcome. In this manner overall cost of the prescriptive and/or phytocannabinoid and/or terpenoid therapeutic regimen can be better managed yet individual patient participation on all levels can be controlled.

In step 607, a smart contract 131 may be created for a selected therapy. A smart contract 131 is a computer protocol intended to digitally facilitate, verify, or enforce the negotiation or performance of a contract. Smart contracts 131 allow the performance of credible transactions without third parties. These transactions are trackable and irreversible. In some embodiments, a patient 101B and/or provider 101A may select a desired therapy that was returned in step 606. The therapy may be provided to the patient 101B and a record of the provision of the therapy may be stored in the blockchain database 113 of the virtual machine engine 151. This record may be used by the virtual machine engine 151 to generate a smart contract 131 associated with the selected therapy provided to the patient 101B which may be stored in a database, such as the blockchain database 113. The virtual machine engine 151 may use data from this contract and one or more client devices 400 to fulfill the terms of the smart contract 131 which may provide compensation to the patient 101B and the one or more providers 101A associated with providing the selected therapy to the patient 101B. In preferred embodiments, a smart contract 131 may be paid for all therapies related to a single ICD-10 code for an agreed upon fixed period of time. In some embodiments, a fixed-fee payment may be received prospectively which would reduce administrative costs for the payer and enable better control of patient by managing adherence, compliance and persistence. Upon completion of step 607, the method 600 may finish 608.

It will be appreciated that some exemplary embodiments described herein may include one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the methods and/or systems described herein. Alternatively, some or all functions may be implemented by a state machine that has no stored program instructions, or in one or more application specific integrated circuits (ASICs), in which each function or some combinations of certain of the functions are implemented as custom logic. Of course, a combination of the two approaches may be used. Moreover, some exemplary embodiments may be implemented as a computer-readable storage medium having computer readable code stored thereon for programming a computer, server, appliance, device, etc. each of which may include a processor to perform methods as described and claimed herein. Examples of such computer-readable storage mediums include, but are not limited to, a hard disk, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory), a Flash memory, and the like.

Embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a tangible program carrier for execution by, or to control the operation of, data processing apparatus. The tangible program carrier can be a propagated signal or a computer readable medium. The propagated signal is an artificially generated signal, e.g., a machine generated electrical, optical, or electromagnetic signal that is generated to encode information for transmission to suitable receiver apparatus for execution by a computer. The computer readable medium can be a machine readable storage device, a machine readable storage substrate, a memory device, a composition of matter effecting a machine readable propagated signal, or a combination of one or more of them.

A computer program (also known as a program, software, software application, application, script, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Additionally, the logic flows and structure block diagrams described in this patent document, which describe particular methods and/or corresponding acts in support of steps and corresponding functions in support of disclosed structural means, may also be utilized to implement corresponding software structures and algorithms, and equivalents thereof. The processes and logic flows described in this specification can be performed by one or more programmable processors (computing device processors) executing one or more computer applications or programs to perform functions by operating on input data and generating output.

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, solid state drives, or optical disks. However, a computer need not have such devices.

Computer readable media suitable for storing computer program instructions and data include all forms of non volatile memory, media and memory devices, including by way of example semiconductor memory devices, e.g., EPROM, EEPROM, and flash memory devices;

magnetic disks, e.g., internal hard disks or removable disks; magneto optical disks; and CD ROM and DVD ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described is this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network or the cloud. The relationship of client and server arises by virtue of computer programs running on the respective computers and having a client server relationship to each other.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequence of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

The computer system may also include a main memory, such as a random access memory (RAM) or other dynamic storage device (e.g., dynamic RAM (DRAM), static RAM (SRAM), and synchronous DRAM (SDRAM)), coupled to the bus for storing information and instructions to be executed by processor. In addition, the main memory may be used for storing temporary variables or other intermediate information during the execution of instructions by the processor. The computer system may further include a read only memory (ROM) or other static storage device (e.g., programmable ROM (PROM), erasable PROM (EPROM), and electrically erasable PROM (EEPROM)) coupled to the bus for storing static information and instructions for the processor.

The computer system may also include a disk controller coupled to the bus to control one or more storage devices for storing information and instructions, such as a magnetic hard disk, and a removable media drive (e.g., floppy disk drive, read-only compact disc drive, read/write compact disc drive, compact disc jukebox, tape drive, and removable magneto-optical drive). The storage devices may be added to the computer system using an appropriate device interface (e.g., small computer system interface (SCSI), integrated device electronics (IDE), enhanced-IDE (E-IDE), direct memory access (DMA), or ultra-DMA).

The computer system may also include special purpose logic devices (e.g., application specific integrated circuits (ASICs)) or configurable logic devices (e.g., simple programmable logic devices (SPLDs), complex programmable logic devices (CPLDs), and field programmable gate arrays (FPGAs)).

The computer system may also include a display controller coupled to the bus to control a display, such as a cathode ray tube (CRT), liquid crystal display (LCD) or any other type of display, for displaying information to a computer user. The computer system may also include input devices, such as a keyboard and a pointing device, for interacting with a computer user and providing information to the processor. Additionally, a touch screen could be employed in conjunction with display. The pointing device, for example, may be a mouse, a trackball, or a pointing stick for communicating direction information and command selections to the processor and for controlling cursor movement on the display. In addition, a printer may provide printed listings of data stored and/or generated by the computer system.

The computer system performs a portion or all of the processing steps of the invention in response to the processor executing one or more sequences of one or more instructions contained in a memory, such as the main memory. Such instructions may be read into the main memory from another computer readable medium, such as a hard disk or a removable media drive. One or more processors in a multi-processing arrangement may also be employed to execute the sequences of instructions contained in main memory. In alternative embodiments, hard-wired circuitry may be used in place of or in combination with software instructions. Thus, embodiments are not limited to any specific combination of hardware circuitry and software.

As stated above, the computer system includes at least one computer readable medium or memory for holding instructions programmed according to the teachings of the invention and for containing data structures, tables, records, or other data described herein. Examples of computer readable media are compact discs, hard disks, floppy disks, tape, magneto-optical disks, PROMs (EPROM, EEPROM, flash EPROM), DRAM, SRAM, SDRAM, or any other magnetic medium, compact discs (e.g., CD-ROM), or any other optical medium, punch cards, paper tape, or other physical medium with patterns of holes, a carrier wave (described below), or any other medium from which a computer can read.

Stored on any one or on a combination of computer readable media, the present invention includes software for controlling the computer system, for driving a device or devices for implementing the invention, and for enabling the computer system to interact with a human user. Such software may include, but is not limited to, device drivers, operating systems, development tools, and applications software. Such computer readable media further includes the computer program product of the present invention for performing all or a portion (if processing is distributed) of the processing performed in implementing the invention.

The computer code or software code of the present invention may be any interpretable or executable code mechanism, including but not limited to scripts, interpretable programs, dynamic link libraries (DLLs), Java classes, and complete executable programs. Moreover, parts of the processing of the present invention may be distributed for better performance, reliability, and/or cost.

Various forms of computer readable media may be involved in carrying out one or more sequences of one or more instructions to processor for execution. For example, the instructions may initially be carried on a magnetic disk of a remote computer. The remote computer can load the instructions for implementing all or a portion of the present invention remotely into a dynamic memory and send the instructions over the air (e.g. through a wireless cellular network or WiFi network). A modem local to the computer system may receive the data over the air and use an infrared transmitter to convert the data to an infrared signal. An infrared detector coupled to the bus can receive the data carried in the infrared signal and place the data on the bus. The bus carries the data to the main memory, from which the processor retrieves and executes the instructions. The instructions received by the main memory may optionally be stored on storage device either before or after execution by processor.

The computer system also includes a communication interface coupled to the bus. The communication interface provides a two-way data communication coupling to a network link that is connected to, for example, a local area network (LAN), or to another communications network such as the Internet. For example, the communication interface may be a network interface card to attach to any packet switched LAN. As another example, the communication interface may be an asymmetrical digital subscriber line (ADSL) card, an integrated services digital network (ISDN) card or a modem to provide a data communication connection to a corresponding type of communications line. Wireless links may also be implemented. In any such implementation, the communication interface sends and receives electrical, electromagnetic or optical signals that carry digital data streams representing various types of information.

The network link typically provides data communication to the cloud through one or more networks to other data devices. For example, the network link may provide a connection to another computer or remotely located presentation device through a local network (e.g., a LAN) or through equipment operated by a service provider, which provides communication services through a communications network. In preferred embodiments, the local network and the communications network preferably use electrical, electromagnetic, or optical signals that carry digital data streams. The signals through the various networks and the signals on the network link and through the communication interface, which carry the digital data to and from the computer system, are exemplary forms of carrier waves transporting the information. The computer system can transmit and receive data, including program code, through the network(s) and, the network link and the communication interface. Moreover, the network link may provide a connection through a LAN to a client device or client device such as a personal digital assistant (PDA), laptop computer, tablet computer, smartphone, or cellular telephone. The LAN communications network and the other communications networks such as cellular wireless and wifi networks may use electrical, electromagnetic or optical signals that carry digital data streams. The processor system can transmit notifications and receive data, including program code, through the network(s), the network link and the communication interface.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A computer implemented method of predicting the health and therapeutic behavior of patients, the method comprising the steps of:
receiving healthcare data of a patient, via a client device, the healthcare data having an existing condition, a new condition, a limiting factor, and a compliance record for the existing condition;
determining, via a computing device processor, a therapeutic behavior pattern of patient using the compliance record for the existing condition;
determining, via the computing device processor, a successful therapy for the new condition based on the therapeutic behavior pattern; and
calculating, via the computing device processor, a cost quote for the successful therapy for a time period based on the limiting factor.

2. The method of claim 1, further comprising the step of determining an unsuccessful therapy for the new condition.

3. The method of claim 2, wherein the step of determining an unsuccessful therapy for the new condition is based on the therapeutic behavior pattern.

4. The method of claim 2, wherein the step of determining an unsuccessful therapy for the new condition is based on the limiting factor.

5. The method of claim 1, wherein the step of determining a successful therapy for the new condition is based on the limiting factor.

6. The method of claim 1, wherein the cost quote is calculated using a coding selected from the group consisting of ICD-10 coding and NDC coding.

7. The method of claim 1, wherein the healthcare data of a patient is received from a blockchain database.

8. The method of claim 7, wherein the healthcare data of the patient is associated with a cryptocurrency token.

9. The method of claim 8, wherein the cryptocurrency token is used as payment for the patient to receive the at least one possible therapy.

10. The method of claim 1, wherein the successful therapy comprises a therapy selected from the group consisting of a laboratory test; a medical device; a digital health technology; a prescriptive drug; medical cannabis; a phytocannabinoid; and a terpenoid molecule.

11. A computer implemented method of providing cost effective therapy for a patient, the method comprising the steps of:
receiving healthcare data of a patient, via a client device, the healthcare data having a new condition;
determining, via a computing device processor, at least one successful therapy for the new condition;
calculating, via the computing device processor, a probability of disease progression for the new condition;
determining, via the computing device processor, at least one possible therapy for the new condition;
calculating, via the computing device processor, a cost quote for the at least one possible therapy; and
creating, via the computing device processor, a smart contract for the at least one possible therapy.

12. The method of claim 11, wherein the cost quote is calculated using a coding selected from the group consisting of ICD-10 coding and NDC coding.

13. The method of claim 11, wherein the healthcare data of a patient is received from a blockchain database.

14. The method of claim 13, wherein the healthcare data of a patient is associated with a cryptocurrency token.

15. The method of claim 14, wherein the cryptocurrency token is used as payment for the patient to receive the at least one possible therapy.

16. The method of claim 11, wherein the at least one possible therapy comprises a therapy selected from the group consisting of a laboratory test; a medical device; a digital health technology; a prescriptive drug; medical cannabis; a phytocannabinoid; and a terpenoid molecule.

17. The method of claim 11, wherein the at least one possible therapy comprises a first possible therapy and a second possible therapy, and wherein the first and second possible therapies are ranked by probability of being a successful treatment.

18. The method of claim 11, wherein the cost quote is calculated for the at least one possible therapy which exceeds a successful probability threshold.

19. The method of claim 11, wherein the smart contract provides compensation to the patient and a provider associated with providing the at least one possible therapy to the patient.

20. The method of claim 11, wherein the smart contract is stored in a blockchain database.
